(19) United States
(12) Patent Application Publication
(10) Pub. No.: US 2016/0174889 A1
(43) Pub. Date: Jun. 23, 2016

(54) Smartphone text analyses

(71) Applicant: Ziv Yekutieli, Zichron Yaakov (IL)

(72) Inventor: Ziv Yekutieli, Zichron Yaakov (IL)

(21) Appl. No.: 14/816,093

(22) Filed: Aug. 3, 2015

Related U.S. Application Data
(63) Continuation-in-part of application No. 14/578,364, filed on Dec. 20, 2014, now abandoned.

(51) Int. Cl. A61B 5/16

(52) U.S. Cl. CPC A61B 5/165

(57) ABSTRACT

The invention comprises systems and methods for the assessment of mental state by means of analysis of key words or phrases written or spoken by a subject. These elements are detected for instance through use of a smartphone app and/or bespoke software keyboard, and their occurrence is logged and analyzed, either locally or remotely. Text and speech may both be analyzed for purposes of the invention, which analyzes key words/phrases for example in terms of their emotional or psychological content, for example counting expressions of positive or negative feelings, attitudes, or other categories. By analysis of frequency of such usages, an estimate or reflection of mental state of the typist is built. Diagnosis of various conditions may thus be carried out, especially by watching how the frequencies and/or percentages of such key elements change over time.

### CLAIM OF BENEFIT

This application claims benefit of U.S. patent application Ser. No. 14/578,364 filed 20 Dec. 2014, being a continuation in part thereof, itself claiming priority from U.S. provisional application 61/928,009 filed 16 Jan. 2014.

### FIELD OF THE INVENTION

The present invention relates to the field of text analyses using a smartphone.

### BACKGROUND OF THE INVENTION

Text entered into search engines, documents, emails, SMS messages and the like may be subjected to analysis by various entities such as service providers, search engines, software developers, and as recent revelations have made apparent, by various domestic and possibly foreign security services.

Analysis would thus appear to be restricted to specific foci such as search interests, domestic terror and the like. There is therefore a lack of tools for extracting general diagnostic information from text, especially that entered into smartphones for various purposes.

### SUMMARY OF THE INVENTION

The invention comprises systems and methods for the detection of key words or phrases. These elements are detected for instance through use of a smartphone application and/or bespoke software keyboard, and their occurrence is logged and analyzed, either locally or remotely. Thus any keystrokes, including those used for searches, writing emails, writing text messages, and the like may all be analyzed by the invention.

The key words/phrases being logged will generally be those having some degree of emotional or psychological content, such as those expressing positive or negative feelings, attitudes, or the like. By analysis of frequency of occurrence of key elements falling into various categories, an estimate or reflection of mental state of the typist can be built. Diagnosis of various conditions may thus be carried out, especially by watching how the frequencies and/or percentages of such key elements change over time.

The foregoing embodiments of the invention have been described and illustrated in conjunction with systems and methods thereof, which are meant to be merely illustrative, and not limiting. Furthermore just as every particular reference may embody particular methods/systems, yet not require such, ultimately such teaching is meant for all expressions notwithstanding the use of particular embodiments.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

The invention analyzes use of key words, phrases, emoticons such as :), ;> and the like, and other expressive elements (hereinafter ‘key elements’) in text or speech, as detected by a smartphone. When (for example) a smartphone user inputs text using the keyboard (or other text input method such as speech-to-text interfaces or the like), this text is analyzed by means of the invention, in terms of use of key elements. The elements are detected for instance through use of an app designed for this purpose, and/or a bespoke software keyboard or other input method. Thus any verbiage, including that used for searches, writing emails, writing text messages, carrying on voice conversations, and the like, is potentially analyzed, be it input as keystrokes, speech, gestures, or the like.

The occurrence of key elements is logged and analyzed, either locally (on the smartphone) or remotely (by a remote server, for instance).

The key words/phrases being logged will generally be those having some degree of emotional or psychological content, such as those expressing positive or negative feelings, attitudes, experiences, or the like. By analysis of frequency of occurrence of key elements falling into various categories, an estimate or reflection of mental state of the typist can be built. Diagnosis of various conditions may thus be carried out, especially by watching how the frequencies and/or percentages of such key elements change over time.

Categories of key elements may simply be defined in terms of lists, for example elements (words, phrases, emoticons, etc.) having positive connotation or negative connotation. Alternatively the key elements may have some value along a discrete or continuous scale, for example classifying elements as falling into one of the ordered categories ‘extremely positive’, ‘positive’, ‘neutral’, ‘negative’, ‘extremely negative’. Key elements may possibly belong to one or more categories such as ‘happy’, ‘engaged’, ‘busy’, and ‘relaxed’.

Whatever the categorization scheme may be, the end result will generally consist of an analysis of the key elements so contrived as to arrive at a quantitative representation of the user's text in terms of its emotional content. This representation may for instance comprise a point in a space having axes such as ‘tension/relaxedness’, ‘fulfillment/frustration’, ‘happiness/depression’ and the like. Other paradigms for such analysis and representation may be used as will be clear to one skilled in the art.

It is within provision of the invention to follow the quantitative representation referred to above over time, in some embodiments to monitor progression of mental state due to disease, medical treatment, or the like. For instance the invention may be used to analyze the progression of Parkinson's or other diseases known to be associated with cognitive and/or emotional impairments.

For this purpose, the complexity of text/speech being used may also be monitored, by analyzing the range of vocabulary, complexity of sentences, variegation of subject matter, and the like. Thus for example sentences being used may be diagrammed and categorized not only in terms of emotional content but in terms of grammatical complexity. The size of the vocabulary and (for example) histograms thereof may be employed. Measures of regularity and deviation therefrom may be employed to gauge the richness of expression. The complexity of linguistic constructs may be estimated by (for example) diagramming the sentences used and determining measures such as average nesting level, depth of embeddings, and the like. These and other measures of the information being communications are taken together to form a diagnostic picture of the mental acuity and emotional state of the subject doing the text entry.

It is within provision of the invention to employ text analysis methods such as latent dirichlet analysis to determine a most-likely topic for every word in a given document (which for the purposes of the invention may consist of an SMS, other text message, email, or the like). These topics and the verbiage used in their discussion may then likewise be rated as before in terms of the emotional content and/or intellectual complexity thereof.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

1. A method for determination of emotional and mental state consisting of:
a. gathering text, speech, and associated input as entered into a smartphone or other digital device;
b. defining a set of key elements having emotional content along one or more scales;
c. analyzing said text, speech, and associated input in terms of said key elements, arriving at a determination of emotional state in terms of the choice and frequency of use of said elements.

2. The method of claim 1 wherein said determination of emotional state is provided in terms of a vector in a space of characteristic dimensions.

3. The method of claim 1 wherein said key elements comprise phrases, emoticons, ‘like’ and ‘dislike’ responses, voice characteristics, speed of speech, and speed of typing.

4. The method of claim 1 further providing an analysis of complexity based on measures of range of vocabulary, complexity of sentences, and variegation of subject matter.

5. The method of claim 1 wherein said analysis is accomplished using latent dirichlet analysis.

6. The method of claim 1, further following differences over time of said mental and emotional state.
